(19) United States
(12) Patent Application Publication
(10) Pub. No.: US 2005/0283070 A1
(43) Pub. Date: Dec. 22, 2005

(54) Systems and methods for qualifying symmetry to evaluate medical images

(76) Inventors: Celina Imielinska, Princeton, NJ (US); Anthony D'Ambrosio, New York, NY (US); Xin Liu, New York, NY (US); Michael Sughrue, New York, NY (US)

Correspondence Address: BROWN, RAYSMAN, MILLSTEIN, FELDER & STEINER LLP, 900 THIRD AVENUE, NEW YORK, NY, 10022, US

(21) Appl. No.: 10/872,666

(22) Filed: Jun. 21, 2004

(51) Int. Cl. A61B 5/05

(52) U.S. Cl. 600/425

(57) ABSTRACT

A method and related software are provided for analyzing images such as computerized tomography images obtained from a standard perfusion CT software package. This method converts image values to relative differences, which represents meaningful side-to-side asymmetry. This conversion may be performed by comparing a small region of the scan to the corresponding region in the contralateral hemisphere, quantifying the degree of relative difference using statistical techniques, and representing this quantity of relative difference in a two dimensional or three diemnsional relative difference map.

The invention relates generally to improved methods and systems for medical and other imaging devices, and more particularly to methods for analyzing electronically acquired image information to determine symmetry and perfusion parameters.

### BACKGROUND OF THE INVENTION

Computed tomography (CT), positron emitted tomography (PET), magnetic resonance imaging (MRI) and other radiological imaging techniques are well known in medical diagnostics. Recent advances in the image processing techniques associated with these technologies has provided medical practitioners with the ability to obtain structural, physiological and functional image data from these tests. The image processing software used in conjunction with MRI and CT allows a user to acquire images of a particular region and process image data to generate physiological image data relating to perfusion parameters.

This perfusion data may be utilized to assess the viability of an area of interest such as certain regions of human tissue by determining various perfusion parameters such as a mean transit time (MTT), a cerebral blood flow (CBF), and a cerebral blood volume (CBV). The image processing software calculates changes in these parameters to generate physiological images of specified regions of human anatomy. Medical practitioners may use these perfusion-weighted images to aid in patient diagnosis by comparing the currently acquired images with any known physiological norms or previous test results to determine any differences.

Currently, medical imaging technologies operate by first generating a grayscale image of the digitally converted signals to construct a pixel-based image of an object of interest. Subsequently, color may be introduced to help highlight areas of varying intensity to facilitate image evaluation. However, image evaluation is a complex process that may be adversely affected by a number factors, such as imperfect images, low resolution images, the limitations of human perception or perceptual bias. Such factors may introduce the possibility that clinical error may occur, which can result in an incorrect patient diagnosis.

In one particular example, Perfusion-Weighted Computed Tomography (CTP) is a relatively recent innovation that utilizes a set of successive axial head CT images to track the time course of signal from an administered bolus of intravenous contrast. These images may be processed using either deconvolution or maximum slope algorithms to extrapolate a numerical value for cerebral blood flow (CBF). While “bolus tracking” methods may provide accurate quantification of CBF under controlled conditions, variability in cardiac function, systemic blood pressure, and cerebrovascular tone often seen in the setting of acute SAH makes quantitative and qualitative assessment of these studies both difficult and potentially hazardous.

While CTP has found some utility in the diagnosis and management of ischemic stroke, its potential use in the diagnosis and management of delayed cerebral vasospasm (CVS) has not been investigated. Furthermore, because this imaging technique is both fast and non-invasive, it is an ideal diagnostic test for this unstable patient population. Unfortunately, due to the inherent variability described above, there is no currently accepted, standardized method of interpreting these scans. Most commonly, scans are interpreted using the qualitative detection of gross side-to-side asymmetry of CBF, an approach that lends itself to misdiagnosis and potential failure to treat CVS. Recent work with CTP has focused on the development of methods to quantitatively analyze CTP images. Most of these approaches utilize the region of interest (ROI) method. In this approach, the clinician circles an ROI on the post-processed CTP image, and the mean CBF is compared to that of the corresponding ROI in the contralateral hemisphere to detect asymmetry. A growing body of data supports improved safety and efficacy of this approach in the setting of acute ischemic stroke.

Accordingly, in view of the foregoing, it would be desirable to provide methods and apparatus for performing electronic image processing that do not rely solely on human experts to evaluate medical images.

It would therefore also be desirable to provide methods and apparatus for electronic imaging that facilitates the assessment of images, and the relative differences between portions of images, and in particular structural, physiological and functional image data, and more particularly for perfusion weighted imaging data, to aid in patient diagnosis.

### SUMMARY OF THE INVENTION

Accordingly, methods and related computational techniques suitable for use in imaging software are provided for evaluating a medical image represented by image data. The method involves assigning an axis or plane of symmetry to the medical image, computing, using the image data, at least one relative difference map based on a comparison of two substantially symmetrical areas around the axis of symmetry, and generating a representation of any relative difference between the two symmetrical areas. In some embodiments, the image data is acquired by scanning a region of interest, such as by performing a computed tomography or other radiological scan of a body.

The axis or plane of symmetry may be assigned by a user through a user interface to the software program, or automatically by the program based on the image data or physical criteria.

The relative difference map may be represented as a two or three dimensional color image illustrating the relative difference between the two substantially symmetrical areas, as a histogram representing the relative difference between the two substantially symmetrical areas, or by any other convenient way to review of the results of the computation.

In some embodiments, the relative difference map is determined by computing a similarity discrepancy between the two substantially symmetrical areas about an axis or plane of symmetry. One known technique useful in performing this statistical calculation is the Kolmogorov-Smimov test. This computation may be performed by first defining at least two windows in the image data, each window representing at least a portion of one of the symmetrical areas for which at least one relative difference map is to be computed. The windows may be defined by positioning each window in substantially equidistant locations from, and positioned symmetrically with respect to, the assigned axis or plane of symmetry. In some embodiments, a composite axis or plane of symmetry (e.g., an average or other composite representation of possible axes or planes) may be used in situations where a single axis or plane is insufficient or does not provide a comprehensive image or the comparison information desired. The windows may be user defined or preset and have n×n pixels of the image data depending on a number of factors such as noise suppression or resolution. In some embodiments, good results are obtained where n=9.

In accordance with some embodiments of the present invention, methods and related computational techniques are provided for analyzing images such as post-processed CTP images obtained from a standard perfusion CT software package, such as the Siemens Medical Solutions package. This method converts CBF values to relative differences, which represent meaningful side-to-side asymmetry. In one embodiment, this conversion is performed by comparing a small region of the scan to the corresponding region in the contralateral hemisphere, quantifying the degree of relative difference, and representing this quantity of relative difference in a two dimensional or three dimensional Relative Difference Map or “RDM.”

In one application, the method involves analyzing the amount of relative difference in both brain hemispheres and six major vascular territories to assess the degree of hypoperfusion in the regions. In this application, a simplified model of the human brain can be defined as a symmetric object if corresponding regions of both hemispheres have comparable structural similarity and CBF equivalence. This model is supported by the following assumptions, made based on widely accepted human brain anatomy and physiology characteristics: (1) In normal cases, the axial CT images of the left and right hemispheres are structurally symmetric and comparable, and there should be no significant relative blood flow difference between the two hemispheres, and (2) In abnormal cases, the left and right hemispheres are still structurally symmetric and comparable, but there is significant relative blood flow difference between the two hemispheres that can be detected using CTP images. The method is preferably automated and may provide a better and more stable analysis of the perfusion parameters of unstable patients such as those with subarachnoid hemorrhage (SAH).

### BRIEF DESCRIPTION OF THE DRAWINGS

The patent or application contains at least one drawing executed in color. Copies of this patent or patent application publication with color drawing(s) will be provided by the Office upon request and payment of the necessary fee.

The above and other advantages of the present invention will be understood upon consideration of the following detailed description, taken in conjunction with the accompanying drawings, in which like reference characters are intended to refer to like parts throughout, and in which:

FIG. 1 is pictorial view of a CT imaging system used in accordance with the principles of the present invention;

FIG. 2 is schematic diagram of the system illustrated in FIG. 1;

FIG. 3 is a representation of a cranial image of a patient, showing the potential location of blockage, generated using the system of FIG. 1;

FIG. 4 is a flow chart representing some of the steps involved in the image comparison method of one embodiment of the present invention;

FIG. 5 is a diagram illustrating a an angle step size relative to maximum ray length suitable for use with the present invention;

FIG. 6 illustrates a close up view of a convex hull and the generic shape constructed in accordance with the principles of the present invention;

FIG. 7 is a graphical representation of the difference between the Fourier shape descriptors for the generic shape and the bounding function;

FIG. 8 illustrates the unwrapped image data plotted on a Cartesian coordinate system;

FIG. 9 shows the data representation of FIG. 8 stretched to uniform radii length in preparation for symmetric analysis;

FIG. 10 is a color computed tomography image of a human brain acquired and constructed with a Siemens Medical Solutions software package;

FIG. 11 is the original gray-scale image use to construct the Relative Difference Map, with the interface shown in FIG. 10;

FIG. 12 is a color relative difference map image generated from the image in FIG. 11, in accordance with one embodiment of the present invention;

FIG. 13 is a histogram that generally represents the areas that relative difference values will tend to populate in patients with varying degrees or symptoms of ischemia;

FIG. 14 is a color relative difference map and associated histograms of the left and right hemispheres of a patient generated in accordance with the principles of the present invention illustrating a relatively normal brain;

FIG. 15 shows the relative difference map illustrated in FIG. 14 with six assigned cranial vascular territories and six associated histograms;

FIG. 16 is a color relative difference map and associated histograms of a patient generated in accordance with the principles of the present invention illustrating an ischemic brain;

FIG. 17 shows the relative difference map illustrated in FIG. 16 with six assigned cranial vascular territories and six associated histograms;

FIG. 18 is a color relative difference map and associated histograms of a patient generated in accordance with the principles of the present invention illustrating a brain with severe vasospasm; and

FIG. 19 shows the relative difference map illustrated in FIG. 18 with six assigned cranial vascular territories and six associated histograms.

### DETAILED DESCRIPTION OF THE INVENTION

Although the present invention is described below in connection with a CT scanning system, it will be understood that the principles and novel concepts described herein may be used with any other magnetic or radiation-based scanning system such as MRI or PET.

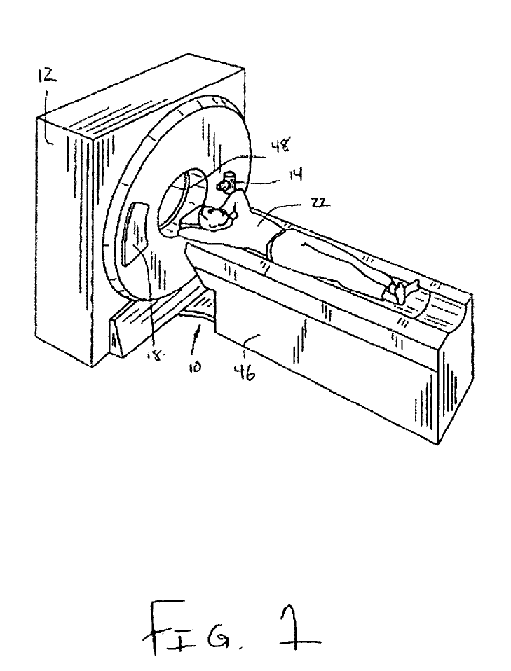

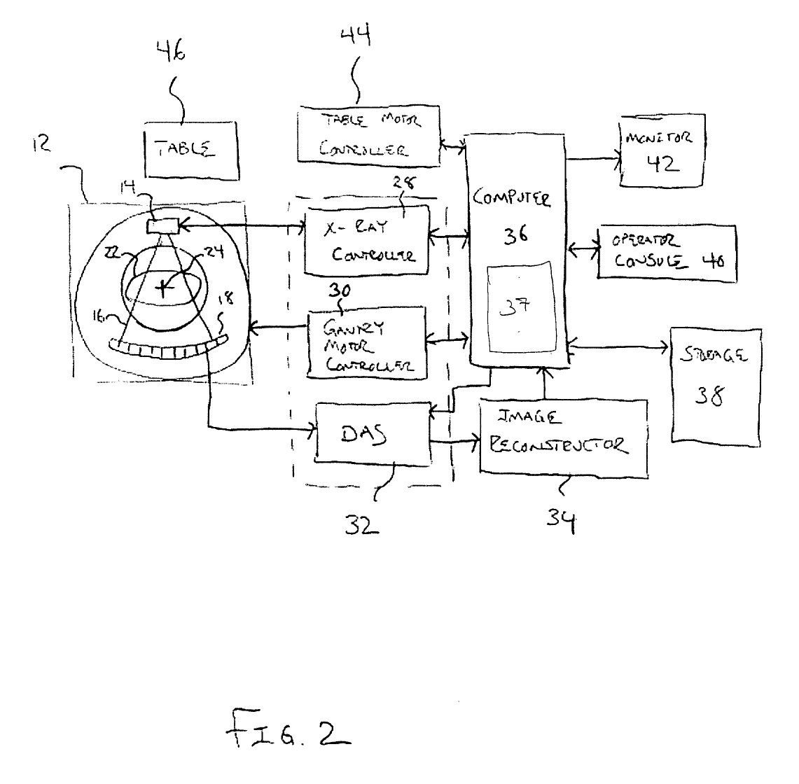

Referring to FIGS. 1 and 2, a CT imaging system 10 is shown as including a gantry 12. Gantry 12 has an x-ray source 14 that projects a beam of x-rays 16 toward a detector array 18 on the opposite side of gantry 12. Detector array 18 is formed by detector elements 20 that together sense the projected x-rays that pass through an object, such as a medical patient 22. Each detector element 20 produces an electrical signal that represents the intensity of an incident x-ray beam and thus the attenuation of the beam as it passes through object or patient 22. During a scan to acquire x-ray projection data, gantry 12 and the components mounted thereon rotate about a center of rotation 24.

As shown in FIG. 2, detector elements 20 may be arranged in one row so that projection data corresponding to a single image slice is acquired during a scan. In alternate embodiments, detector elements 20 are arranged in a plurality of parallel rows, so that projection data corresponding to a plurality of parallel slices can be acquired simultaneously during a scan (which may be subsequently processed into three dimensional images).

Rotation of gantry 12 and the operation of x-ray source 14 are governed by a control mechanism 26 of CT system 10. Control mechanism 26 may include an x-ray controller 28 that provides power and timing signals to x-ray source 14 and a gantry motor controller 30 that controls the rotational speed and position of gantry 12. A data acquisition system (DAS) 32 in control mechanism 26 samples data from detector elements 20 and converts the data to digital signals for subsequent processing. An image reconstructor 34 receives sampled and digitized x-ray data from data acquisition system 32 and performs high speed image reconstruction. The reconstructed image is applied as an input to a computer 36 that stores the image in a storage device 38.

Computer 36 also receives commands and scanning parameters from an operator via console 40 that includes a data input device (not shown). An associated display screen 42 allows the operator to observe the reconstructed image and other data from computer 36. Operator supplied commands and parameters are used by computer 36 to provide control signals and information to data acquisition system 32, x-ray controller 28 and gantry motor controller 30. In addition, computer 36 may operate a table motor controller 44 which controls a motorized table 46 to position patient 22 in gantry 12. In particular, table 46 may move portions of patient 22 through gantry opening 48.

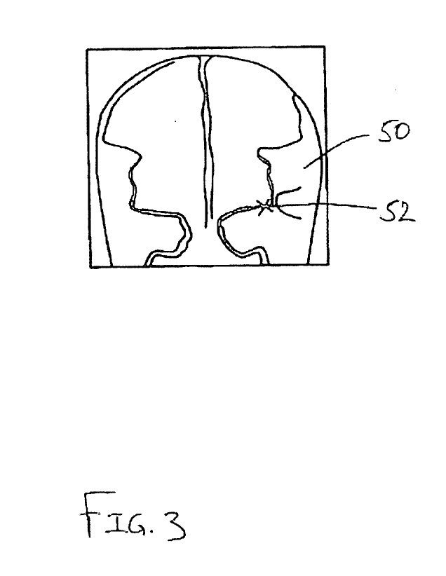

As shown in FIG. 3, blood flow parameters of an organ of patient 22 such as brain 50 are measured by injecting a contrast substance (e.g., one containing iodine) into patient 22 that produces a contrasting appearance on CT images. System 10 may acquire multiple images of the same section of interest with a fast CT scanner that allows for tracking of blood flow in the human brain. Blood flow, blood volume, and mean transit time can be measured by examining the perfusion of the contrasting substance, which in some cases allows blockages 52 to be recognized. Such a blockage may be recognized by a change in color indicating a change in volume, flow, or mean transit time.

In some embodiments of CT scanner 10, maps with different color schemes are used for CT perfusion parametric images to facilitate their assessment. For example, display 42 may be a color display, and software in computer 36 uses a mapping of intensities to color to enhance images for analysis. The computational technique described herein may be used to compare reconstructed images denoting the same region at different times, the differences in measurements, as a function of time, of quantities such as blood flow, blood volume, and mean transit time for blood containing the injected contrast to move through vessels. Depending upon the intended purpose of the images selected for display, intensities or intensity differences may be mapped onto a set of colors by the software and the images are displayed on color display 42.

The colors representing intensities (or intensity differences) may be mapped using threshold values that correspond to a physiological threshold. For example, to facilitate detection of tissues in which an ischemia is occurring, a predetermined color, such as blue, is mapped to threshold levels selected to show where the ischemia is occurring. On the other hand, other colors such as green are mapped to threshold areas of lower intensity in a reconstructed image representing healthy tissues, and another color such as red may be mapped to areas having intensities characteristic of blood vessels. Such mappings are useful for assessment of blood flow.

Once parametric and image information have been captured as described above, subsequent data processing may be performed in computer 36 (using symmetry analysis software 37 constructed as described herein) for subjects which have symmetrical or substantially symmetrical counterparts such as human anatomy or other naturally occurring symmetrical objects. This processing may produce, among other things, an asymmetry determination and relative difference maps (described in more detail below) that illustrate relative differences between the two objects with a high degree of precision and may be used to supplement or confirm any qualitative symmetry analysis performed by a human medical specialist.

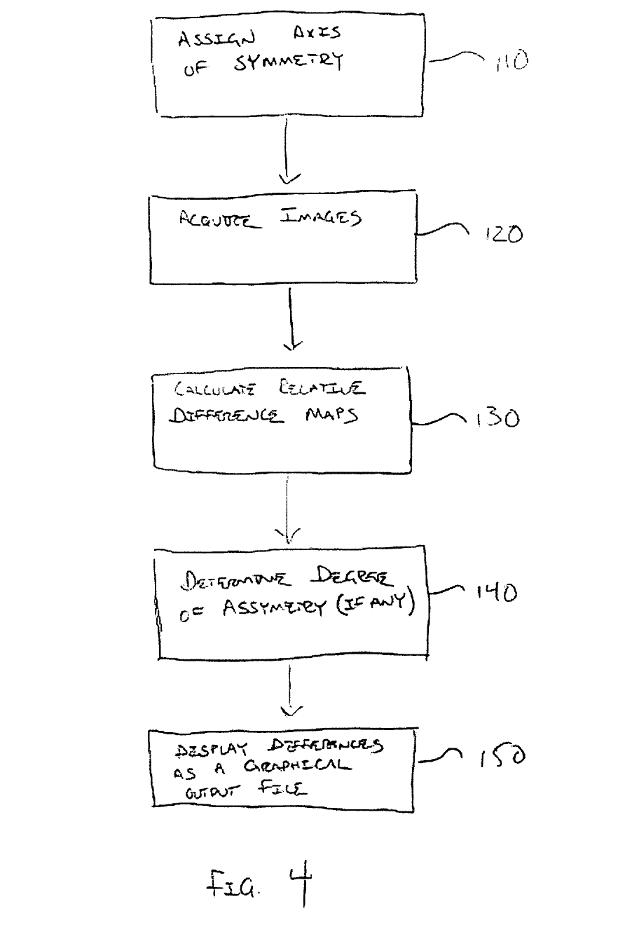

In general, the method may include five steps that are shown in the flow chart of FIG. 4. As shown, step 110 involves assigning a plane of symmetry on a subject to be scanned. In some embodiments, this step may be performed before the image capture process begins so the subject can be properly aligned within the scanning mechanism (such as CT system 10). This helps eliminate the capture of undesired asymmetric data and thereby reduces clinical error. In other embodiments, however, an entire region may be scanned first, and the plane of symmetry may be assigned later based on the acquired data or on an area of interest. Often, the plane of symmetry is chosen such that the subject is separated into two distinct symmetrical regions. The plane of symmetry may be assigned by comparison software resident in computer 36 based on certain physical or other known orientation criteria.

At this point, the scanning system, such as scanning system 10, acquires the necessary perfusion-weighted images (step 120) and prepares the acquired data for post-processing. This step may be performed by software in a Siemens Medical Solutions package. Next, at step 130, an axis of symmetry may be assigned and a mathematical analysis is used to compare the selected regions with respect to the assigned axis of symmetry in accordance with the principles of the present invention. The mathematical analysis may include: 1) computing the convex hull of the input image and its corresponding Fourier shape descriptor, 2) computing a series of centroids in the convex hull that may define the axis of symmetry; 3) “unwrapping” the convex hull over a rectangular map with the axis of symmetry in the middle (converting the convex image to a substantially rectangular or square one, similar to creating a Mercator projection of the convex hull), 4) optionally normalizing the unwrapped image, 5) analyzing pixel by pixel symmetry using binary and/or gradient image data, and 6) analyzing and quantifying the degree of symmetry (e.g., with RDMs, histograms, etc). This is discussed in more detail below.

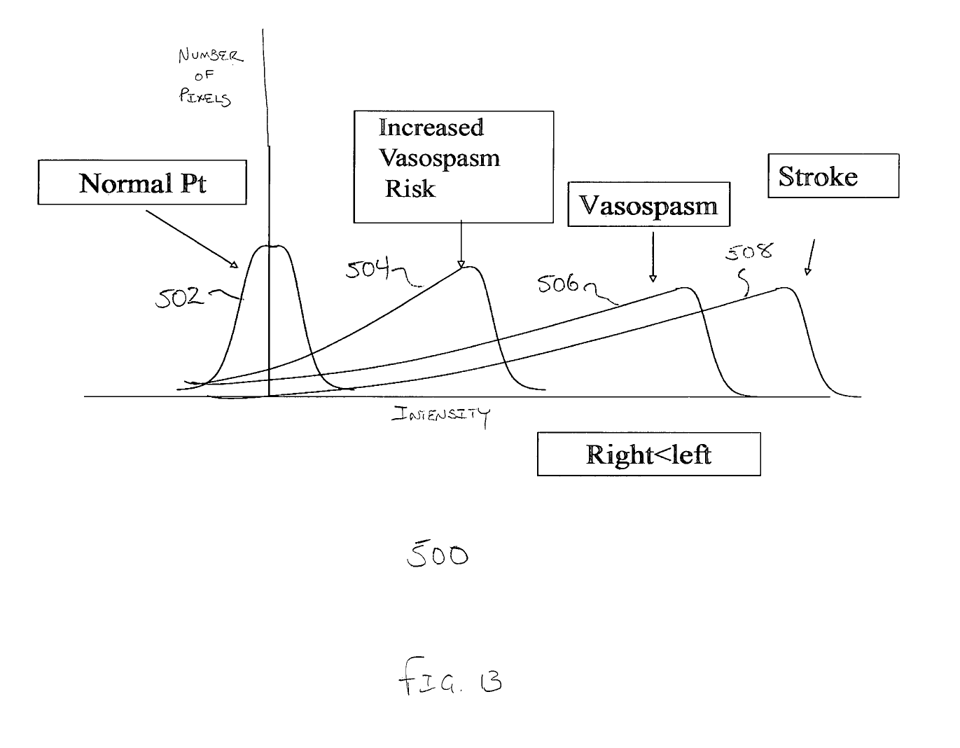

The regions to analyze may be selected by a user based on a particular interest, and the analysis may be performed iteratively for varying parts of the region or for the image in its entirety taken region by region. The mathematical analysis may include any method that determines the difference between two populations of data points, although a method that does not require a normal distribution of data points is preferred. Each value that appears on the RDM indicates a point of asymmetry. Any detected asymmetries may be plotted in two and/or three dimensional maps with results also produced in the form of relative difference maps or other representations having a similar functionality. At step 140, the degree of asymmetry in specific regions of interest may be quantified and analyzed mathematically by generating histograms, for example, that plot the number and frequency of differential points between the selected regions (shown in FIG. 13, and discussed in more detail below). At step 150, the quantitative analysis performed in step 140 may be linked to a specific output format suitable for the desired application. Such an application may include a graphical display program that illustrates the asymmetries present in portions of a human body.

In the preferred embodiment, the above described steps are carried out automatically through software routines in a computer such as computer 36. This automated image acquisition and comparison method can be used to aid or supplement the assessment of symmetry (or asymmetry) of an object by a human specialist whose analytical ability is limited by the boundaries of human acuity. Thus, the method provides a medical practitioner with the detailed information necessary to make correct and accurate diagnosis decisions even when the symptoms are beyond what would normally be noticed by a human observer. It also provides the medical community with a computer assisted diagnostic tool to improve diagnostic decisions and a teaching and training tool to help medical students recognize symmetries or asymmetries in patients.

In one embodiment of the present invention, computation of the convex hull described above may be determined through the use of bounding functions. For example, to obtain a function that bounds a region in an image described by points on the outer most edge of the region, we may generate rays from a centroid (cₓ, c_{y}) of the region outward at specific increasing angles theta. To obtain a valid sampling, we increase theta at each step as indicated below in equation 5:

$$Δθ < {sin}^{- 1} ( \frac{1}{2 {R}_{max}} ) \qquad ( 5 )$$

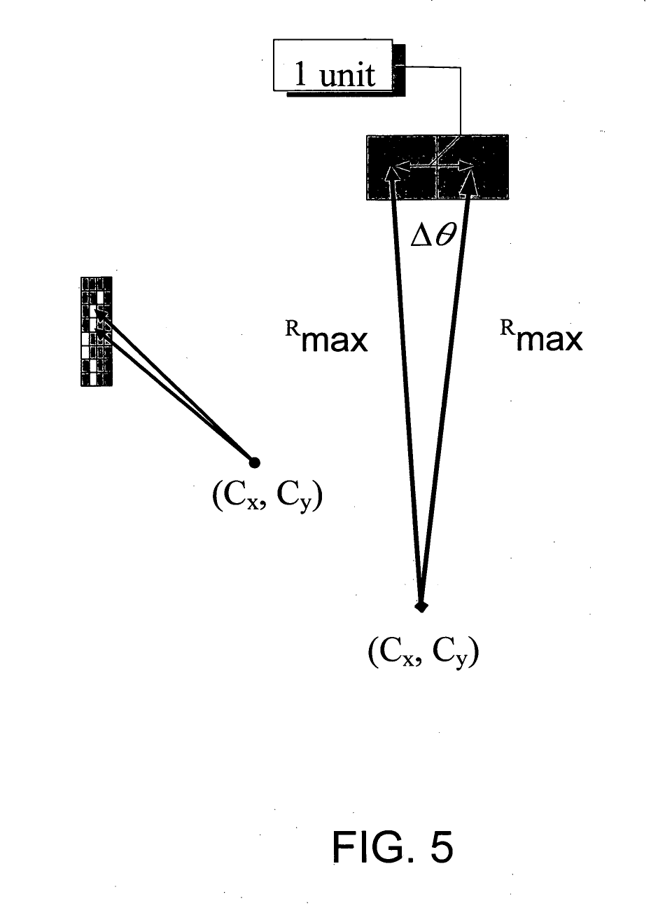

This is shown in FIG. 5.

Some rays may pass through a singularity in the boundary, meaning, it is possible, although unlikely, that a ray will pass between two boundary pixels that are diagonally connected. To prevent this from happening we can make the ray have a thickness on the order of the precision of CT system 10.

By recording the length of the ray at each angle, we have a description of a bounding function R(θ) of the region as a function of theta. This function may not have desirable properties (e.g., it not may not be convex).

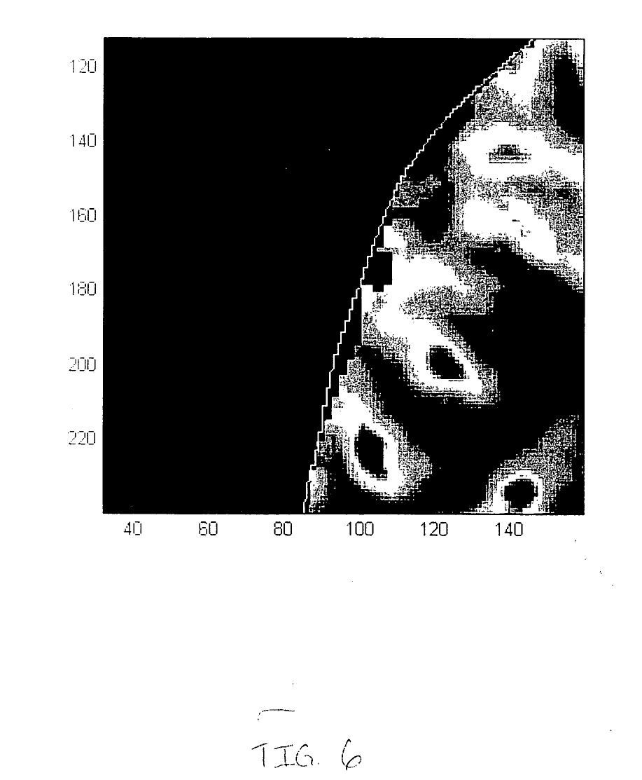

The convex hull of the boundary can be obtained, with the points on the convex hull being parameterized by theta. By linearly interpolating the radii between two points on the convex hull, it is possible to obtain a “generic shape” that shares points with the convex hull, but smoothly varies from on point to the next, (which may not be convex). This is shown in FIG. 6.

This function provides a way to determine if a point within a distance of Rₘₐₓ on the centroid is inside or outside of the region enclosed by the bounding function. This may be accomplished by calculating the angle, and comparing the two distances obtained.

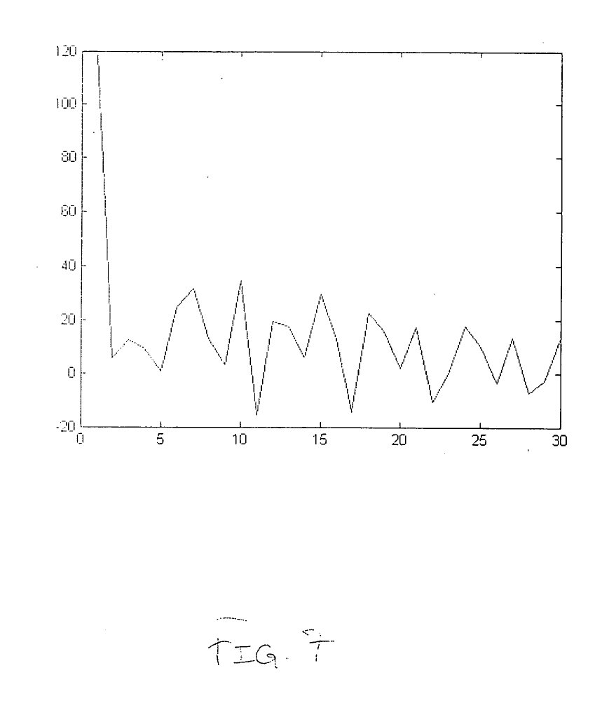

After the periodic bounding function is obtained, Fourier Shape Descriptors of the bounding function may be calculated as well as the centroids of any angular section of the object. The difference in the shape descriptors for the generic shape and the bounding function to determine the stopping point of the rays and therefore the shape of the convex hull. This is generally shown in FIG. 7.

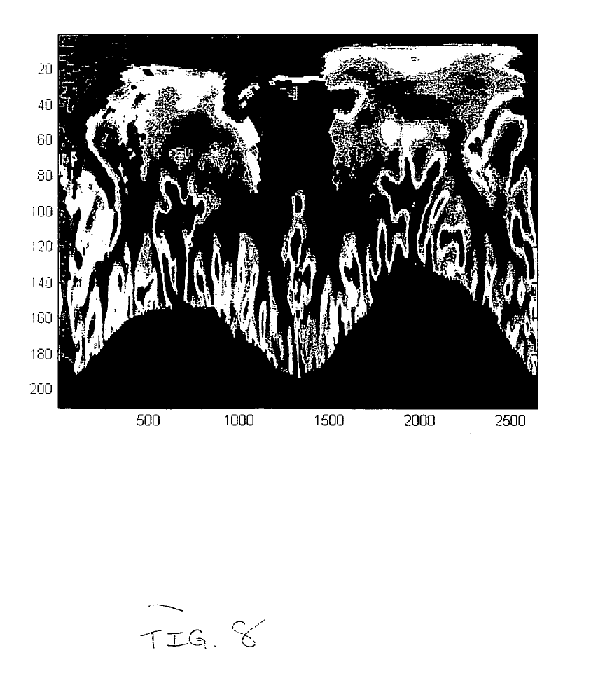

At this point the data may be unwrapped by converting the convex image to a substantially rectangular image and each of the radii can be renormalized to equal length, to facilitate the comparison of features in the left and right halves of the image (shown in FIG. 8).

This procedure may be generalized to three dimensions, where the radius is a function of the solid angle,

$$r ( θ , ϕ ) , 0 \leq θ < 2 π , - \frac{π}{2} \leq ϕ \leq \frac{π}{2} ( duplicated points at - \frac{π}{2} , \frac{π}{2} )$$

are removed later. Extracted from these radii, and the “general shape” is constructed.

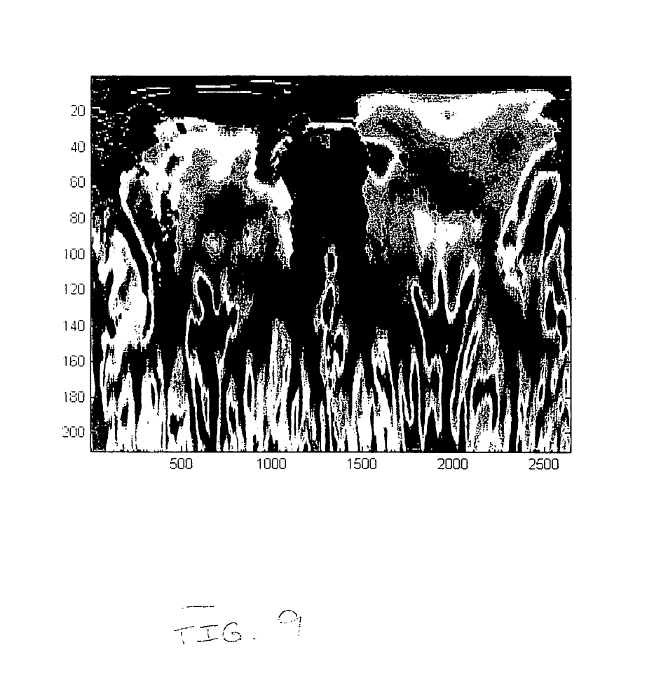

By iterating this technique with decreasing values of Rₘₐₓ, sets of radii may be constructed that can be combined to obtain the boundary of the object parameterized by arc length, extending the object to boundaries that are not a function of theta (shown in FIG. 9).

Although the above described method has applicability to virtually any substantially symmetrical object, the principles of the present invention are well suited for determining the presence of ischemia in the regions of the human body such as the brain. For example, it can be shown using the so called “maximum slope method” that CBF at any location in the brain may be determined by observing the maximum slope of C(t) at a particular location and dividing by the difference of Cₐ(t) at the input (e.g., anterior cerebral artery) and the output Cᵥ(t) (e.g., superior sagittal sinus). This provides the following relationship:

$$( F / V ) = \frac{{( ⅆ C / ⅆ t )}_{tissue} ( {t}_{max\_slope} )}{( {C}_{a} ( {t}_{max\_slope} ) - {C}_{v} ( {t}_{max\_slope} ) )} \qquad ( 1 )$$

Thus, it can be seen from the relationship in equation 1 that the maximum slope for any tissue is achieved at the same time when the input slope Cₐ(t) reaches its peak. Consequently, CBF may be calculated at any location by tracing the maximum slope and dividing it by the maximum value intensity value at the anterior cerebral artery.

By observing Cᵥ(t) curve to compute its maximum, CBF and CBV may be derived using the following relationships:

$$\begin{matrix}CBF ( any\_location ) = \frac{max\_slope ( any\_location )}{max\_value ( {C}_{v} ( t ) )} & ( 2 ) \\ CBV ( any\_location ) = \frac{max\_value ( any\_location )}{max\_value ( {C}_{v} ( t ) )} & ( 3 )\end{matrix}$$

Furthermore, if Cₐ(t) and Cᵥ(t) curves are obtained independently and then are superimposed on one another, it is possible to asses different cardiac output. However, because the “relative” values of CBF and CBV are considered to be more reliable than the absolute values of these quantities, the difference maps and relative difference maps may be calculated as described below.

Difference maps may be calculated by subtracting the pixel illumination value from one scanned hemisphere with those found on the contralateral hemisphere. For example, values for CBF may be calculated for both sides and compared. An ischemia score may be assigned on the pixel differential if significant CBF is detected.

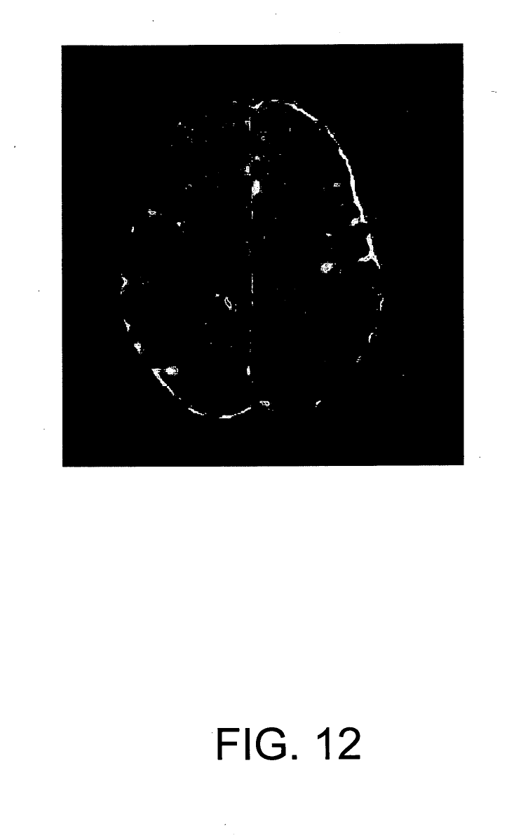

Relative difference maps may be obtained by comparing the pixel values of each hemisphere and computing the ratio of pixels with a lower CBF score to those with a higher one. The relative difference map may be displayed as a color differential map highlighting areas of ischemia or reduced blood flow for consideration by a medical specialist (shown in the color illustration in FIG. 12).

The following provides a general list of conditions that may be employed by comparison software in computer 36 to generate difference maps and relative difference maps for display to a user.

1. Difference Maps: DM-CBV, and DM-CBF.

(a) L2R DM (left to right difference map)
   if l2r<0→display its absolute difference on the L side (pixel differential)
   if l2r>=0→display Black (zero intensity)
(b) R2L DM (right to left difference map)
   if r2l<0→we display its absolute difference on the R side (pixel differential)
   if r2l>=0→display Black (zero intensity)
      2. Relative Difference Maps: RDM-CVF, and RDM-CBF.
a) L2R DM
   if l2r<0→compute the ratio of the absolute difference (pixel differential) divided by the intensity on the RIGHT (the good one) hand side, and display it on the LEFT hand side
   if l2r>=0→display Black (zero intensity)
(b) R2L DM
   if r2l<0→compute the ratio of the absolute difference (pixel differential) divided by the intensity on the LEFT (the good one) hand side, and display it on the RIGHT hand side
   if r2l>=0→display Black (zero intensity)
      3. Relative Maps: RM-CVF, and RM-CBF. In relative maps we relate the intensity on the BAD side (the one with the lower CBF value) to the intensity on GOOD side, (the one with the higher CBF value) this will allow us the “intervals” for normalized relative values.
a) L2R DM
   if l2r<0→compute ratio of intensity on the LEFT hand side (the bad one) divided by the intensity on the RIGHT (the good one) hand side, and display it on the LEFT hand side
   if l2r>=0→display Black (zero intensity)
(b) R2L DM
   if r21<0→compute ratio of intensity on the RIGHT (the bad one) divided by the intensity on the LEFT (the good one) hand side, and display on the RIGHT hand side
   if r2l>=0→display Black (zero intensity)
      For TTP, we do the reverse:
1. Difference Maps: DM-TTP.
(a) L2R DM
   if l2r>0→display its absolute difference on the L side (pixel differential)
   if l2r<=0→display Black (zero intensity)
(b) R2L DM
   if r2l>0→display its absolute difference on the R side (pixel differential)
   if r2l<=0→display Black (zero intensity)
2. Relative Difference Maps: RDM-TTP.
a) L2R DM
   if l2r>0→compute the ratio of the absolute difference (pixel differential) divided by the intensity on the LEFT hand side (the bad one) hand side, and display it on the LEFT hand side, too
   if l2r>=0→display Black (zero intensity)
(b) R2L DM
   if r2l>0→we compute the ratio of the absolute difference (pixel differential) divided by the intensity on the RIGHT (the bad one) hand side, and display it on the RIGHT hand side
   if r2l<=0→display Black (zero intensity)
3. Relative Maps: RM-TTP. Compute the ratio of the “good side” the opposite to the “bad one”.
a) L2R DM
   if l2r>0→compute ratio of intensity on the RIGHT hand side (the good one) divided by the intensity on the LEFT (the bad one) hand side, and display it on the LEFT hand side
   if l2r<=0→display Black (zero intensity)
(b) R2L DM
   if r2l>0→we compute ratio of intensity on the LEFT (the good one) divided by the intensity on the RIGHT (the bad one) hand side, and display on the RIGHT hand side
   if r2l<=0→display Black (zero intensity)

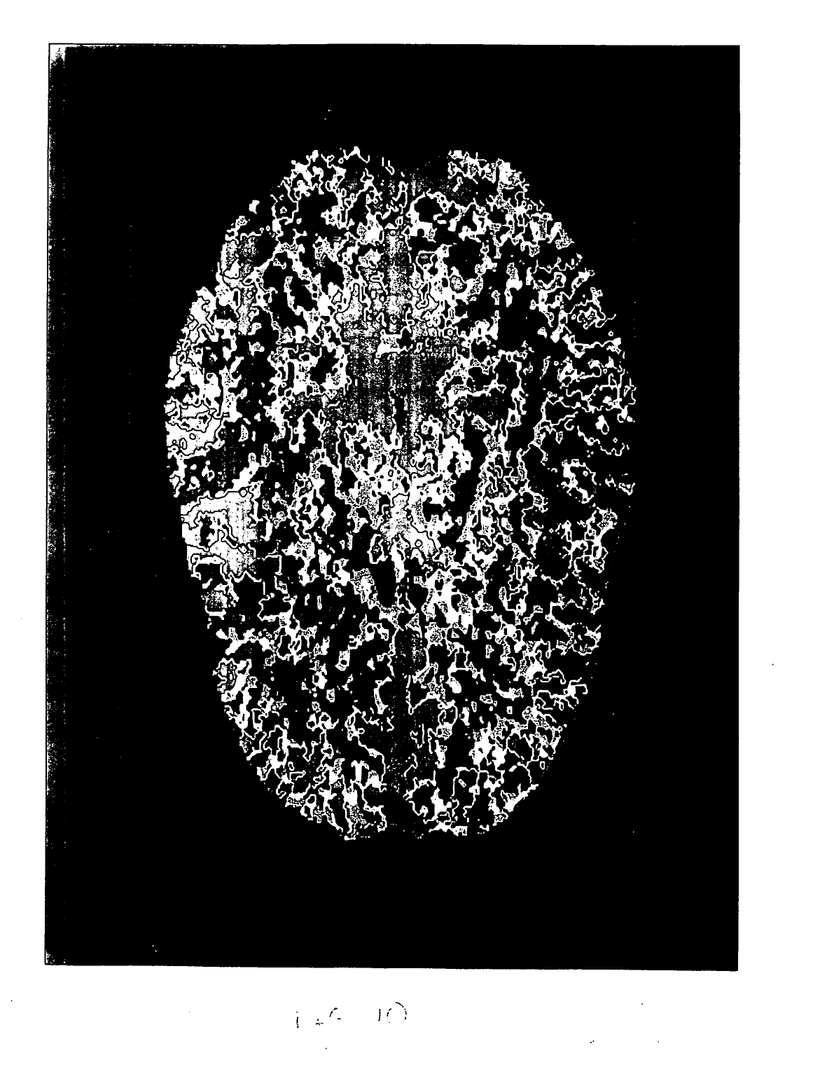

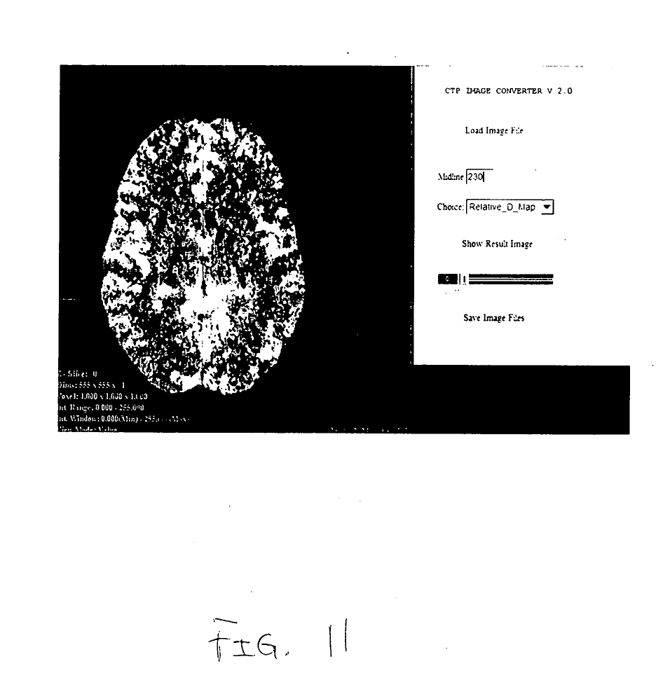

In operation, using the above guidelines, system 10 may acquire a number of CT images to create the grayscale CBF image of a human brain shown in FIG. 11. This image may be constructed using system 10 and known CT perfusion software such as that currently available from Siemens corporation. This grayscale image may be transformed into a predetermined binary format such as an eight bit digital word so the scales in the original image may be normalized into range from zero to two hundred fifty five, which may be used to assign colors to the image. This is shown by the color image in FIG. 10. Although an eight bit words are suitable for some embodiments of the present invention, it will be understood that words of a different size may be used to obtain images with a greater or lesser degree of precision if desired.

Next, a axis of symmetry may then be estimated (or computed) as a straight line drawn along the anterior-posterior axis through the septum pelucidum to equally divide the brain image shown in FIGS. 10-11 into two substantially symmetric hemispheres (right and left). This operation may be performed automatically in accordance with the present invention by comparison software in computer 36, or selected manually by the medical practitioner performing the imaging task.

Next, to quantify the symmetry of the scanned image, the comparison software performs a statistical discrepancy test to determine the difference between the observed and expected cumulative frequencies between the data points acquired from the symmetric hemispheres. One such test suitable for this operation is the Kolmogorov-Smimov test which is a non-parametric statistic test that does not require the acquired data points to be normally distributed as is the case in Gaussian based methods. One skilled in the art will recognize that other statistical tests may be used for this analysis. This test is based on the empirical distribution function as defined in equation 4, given N ordered data points Y₁, Y₂, . . . , Y_{N}
E_{N}=n(i)/N (4)
where n(i) is the number of points less than Yi and the Yi are ordered from smallest to largest value. This step function increases by 1/N at the value of each ordered data point. Using this formula, the statistically significant differences between the two populations may be determined. This is preferably accomplished in accordance with the principles of the present invention by scanning each hemisphere into a number of overlapping symmetric sections or “windows” which are compared against one another (from opposite hemispheres) to determine the absolute difference between the two. The size of the windows may vary depending on the size of the converted digital word or may be adapted to achieve a particular diagnostic goal. In one embodiment as represented by the algorithms described herein, a nine by nine pixel window is used. It has been found that such a window provides good resolution as compared to the noise generated by small numbers of anomalous pixels. However, other window sizes may be used if desired.

To determine asymmetries between the areas covered by the windows, the average intensities of pixels in one window from one hemisphere are subtracted from those of the contralateral hemisphere, and the absolute difference is divided by the intensity value on the side where CBF reading is relatively larger and higher (“relatively normal hemisphere”). The result is displayed on the side where the reading of the mirrored window is smaller (in the case of CBF and CBV parameters) or larger (in the case of TTP parameters) (“relatively abnormal hemisphere”) to display the score for the relative difference map. A relative difference map of the image depicted in FIG. 11 constructed in accordance with the present invention is shown in FIG. 12.

Further analysis of the relative difference map shown in FIG. 12 may be performed by subdividing each hemisphere into six major cerebrovascular territories and calculating statistical difference charts, such as histograms, to illustrate (an subsequently analyze) the degree of difference between the two hemispheres. The degree of difference in each of the territories will be indicative of the type and degree of problem the patient is experiencing. As the peak of the curve moves further to the right, representing a greater degree asymmetry, the severity of the problem increases. For example, as shown in histogram 500 of FIG. 13, curve 502 represents an expected distribution of relative difference values in a region of a brain that shows substantially normal blood flow distribution. Moving to the right, curve 504 represents an expected distribution of relative difference values in a region of a brain that shows a blood flow distribution indicating an increased risk of vasospasm. Moving further to the right, curve 506 represents an expected distribution of relative difference values in a region in a brain that shows a blood flow distribution indicating an existing vasospasm. At the far right, curve 508 represents an expected distribution of relative difference values in a region in a brain that shows a blood flow distribution indicating a patient with an existing infarct.

Thus, as can be seen from the above, the invention provides a way in which brain asymmetry may be quantified and analyzed and used as a diagnostic tool to recognize or predict brain disease. By comparing successive histograms 500, for example, a medical practitioner may diagnose a slight brain condition that normally may go unnoticed, diagnose an existing brain disease with certainty, or by monitoring the progress of the peak of the curves on histogram 500, recognize a trend or a degenerating state. This is an advantage over other existing techniques that merely display an image of the brain with color perfusion parameters indicative of blood flow that have to be manually compared and diagnosed. The quantification offered by the present invention should ideally be used to supplement existing diagnostic techniques.

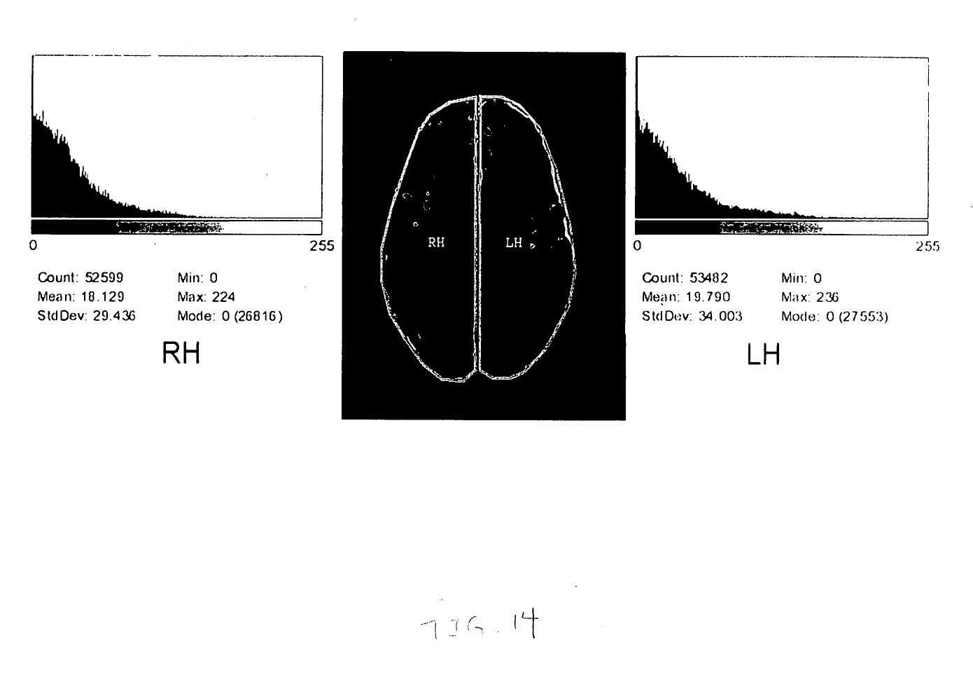

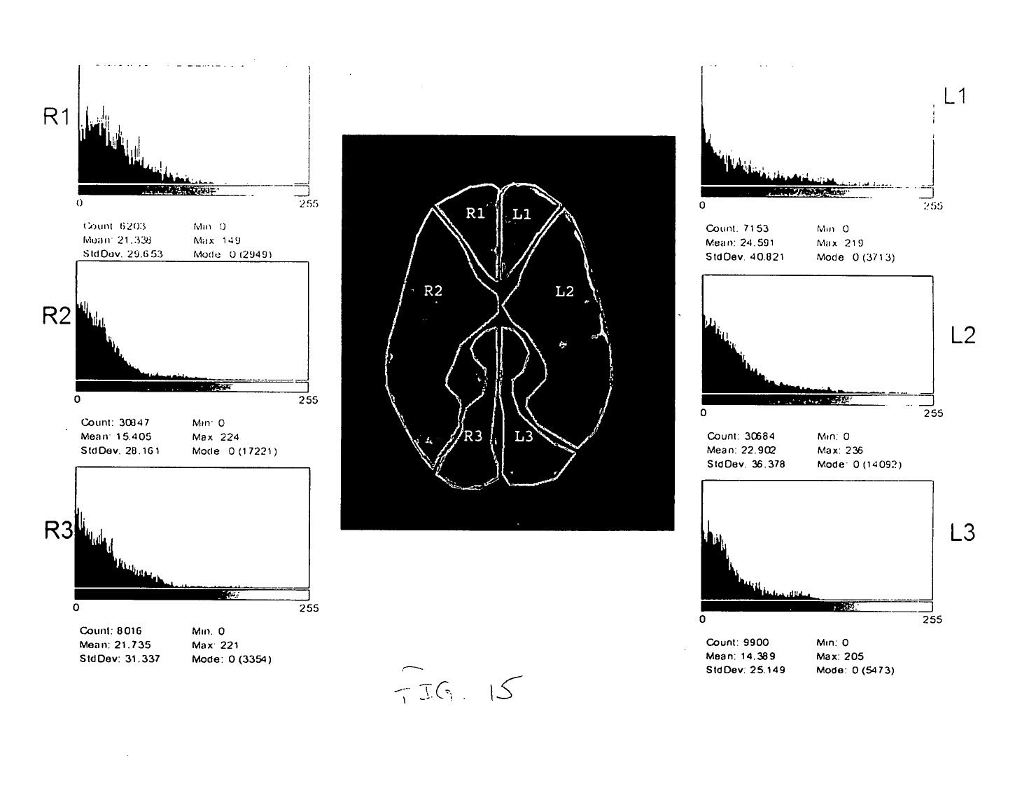

Examples of relative difference maps and histograms generated in accordance with the present invention are shown in the color images of FIGS. 14-19. FIG. 14 illustrates a relative difference map in the center with histograms RH and LH comparing each brain hemisphere. The patient who generated this data was a male in his early forties with a Hunt and Hess grade 2 SAH. Cerebral angiography disclosed a large (2 cm) MCA aneurysm, which involved the lenticulostriates. The patient's clinical course was unremarkable from a neurological standpoint, with no episodes of CVS detected by daily Transcranial Doppler sonography (TCD), cerebral angiography, or routine neurological examination. On SAH Day 5, the patient underwent CTP scan, data from which was processed as disclosed herein. The relative difference maps and histograms generated are depicted in FIGS. 14-15. FIG. 15 shows how the six vascular cranial territories were assigned to the relative difference map in the center with: L1 representing the area including the left anterior cerebral artery, L2—representing the area including the left middle cerebral artery, L3—representing the area including the left posterior cerebral artery, R1—representing the area including the right anterior cerebral artery, R2—representing the area including the right middle cerebral artery, R3—representing the area including the right posterior cerebral artery (although it will be understood the these regions may be rearranged or changed to serve different diagnostic goals). As can be seen from the regional histograms in FIG. 15, the vascular territories demonstrate a relatively minimal deviation of the curve from zero in all territories, indicating relatively normal levels of perfusion throughout the brain.

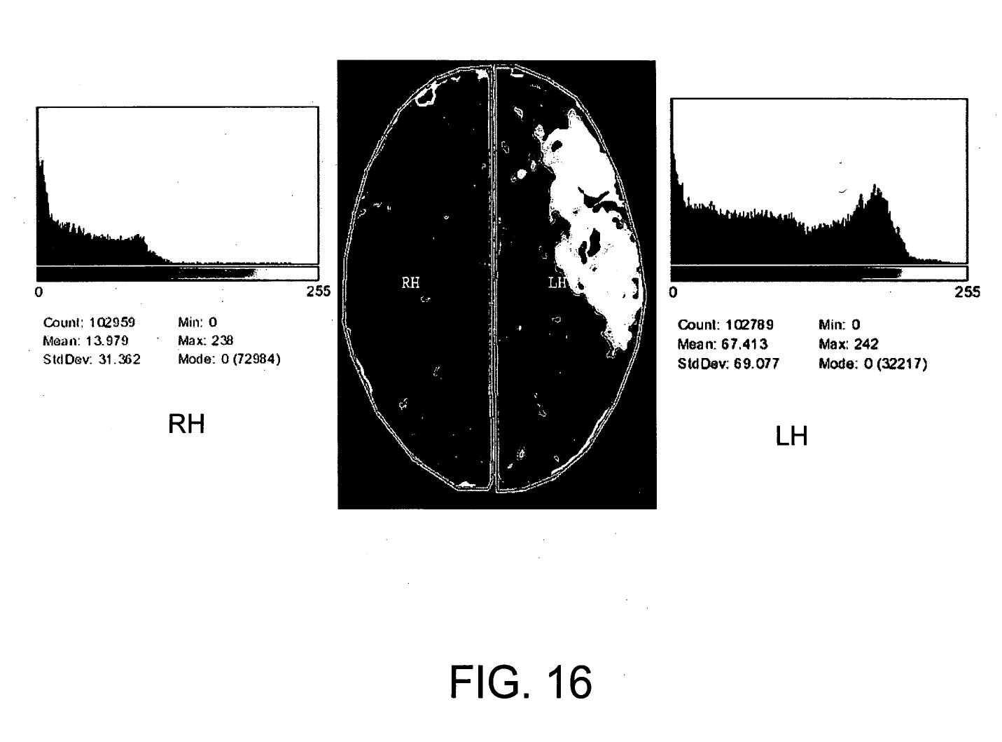

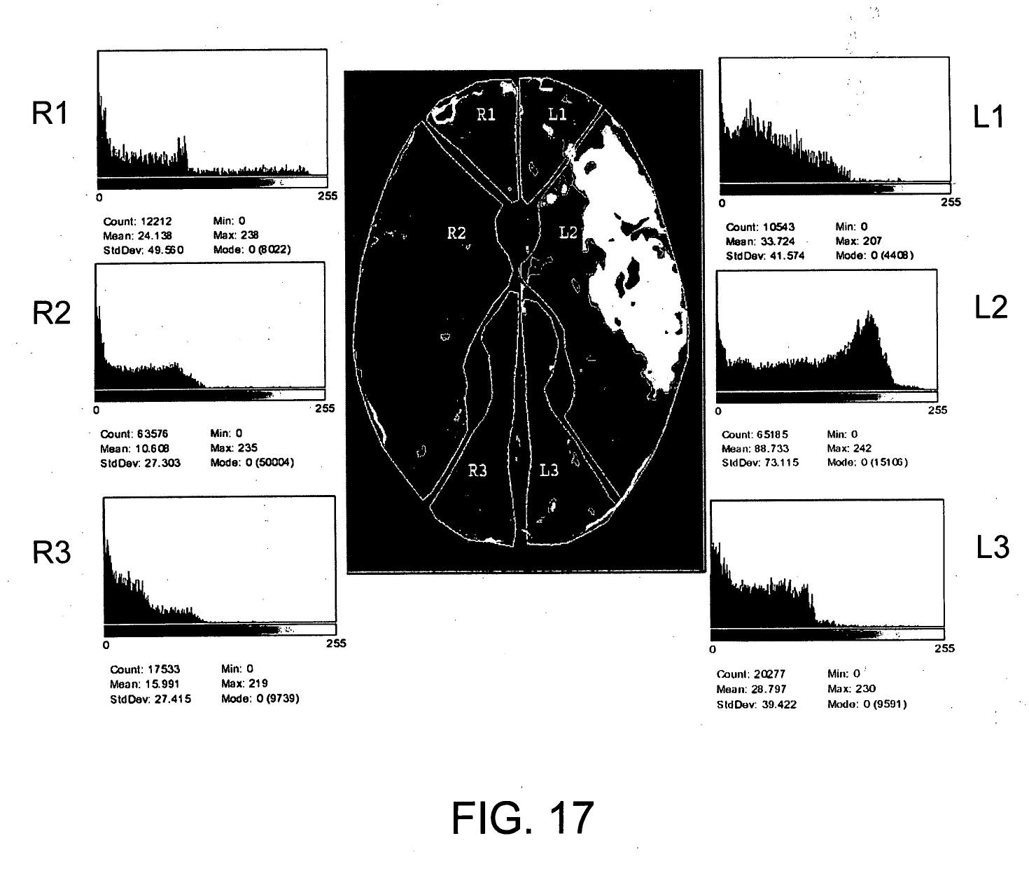

The patient who generated the data shown in FIGS. 16 and 17 was a female in her late seventies who was presented with symptoms consistent with left MCA infarction. The right side of the relative difference map in FIG. 16 (left side of the brain) clearly demonstrates large wedge shaped region of severe hypoperfusion in the MCA territory consistent with acute proximal MCA occlusion. When this image has been processed using the methods described herein, a clear peak on the far right is seen in the LH histogram (FIG. 16) representing the left MCA territory that is consistent with our theoretical stroke curve 508 as shown in FIG. 13. The histograms for other vascular territories (L1 and L3 and R1-R3) in FIG. 17 have significantly smaller means, and many appear relatively “normal.”

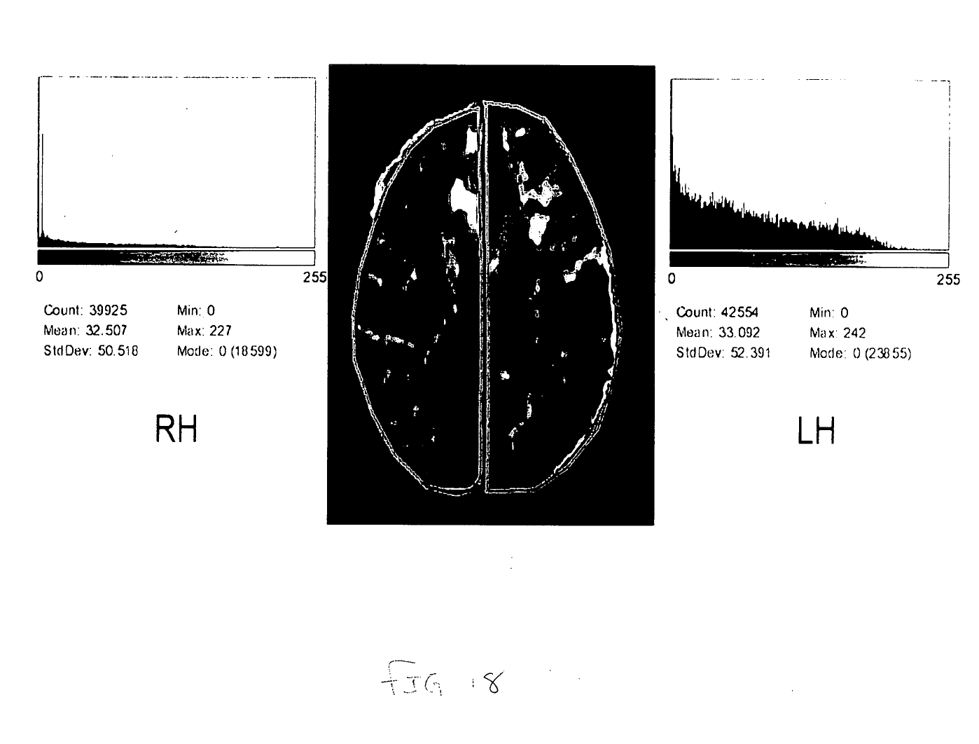

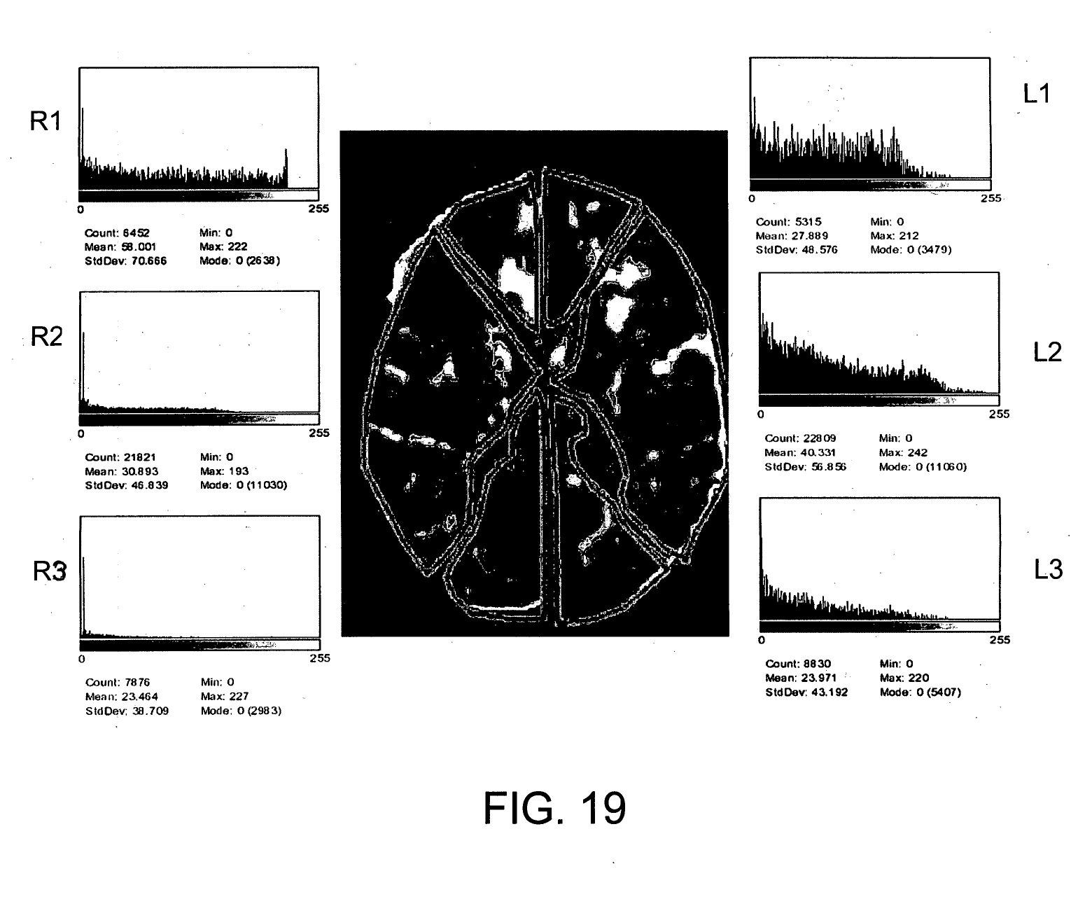

The patient who generated the data shown in FIGS. 18 and 19 was a woman in her mid thirties who was presented with Hunt and Hess grade 4 SAH. Cerebral angiography disclosed a 4 mm×3 mm right anterior choroidal artery aneurysm. Her neurological examination improved significantly postoperatively. However, on SAH day 5, she experienced an acute decline in mental status, however neurological exam demonstrated no focal neurological deficit. The patient subsequently developed bilateral arm weakness and was taken for angiography, which revealed severe vasospasm of the right and left MCA and right ACA. This spasm was treated with angioplasty, with significant clinical improvement. Unfortunately however, follow-up MRI two months later demonstrated old cerebral infarction in the right frontal lobe. The relative difference map of FIG. 19 demonstrates significant regions of side-to-side asymmetry in the Left MCA and Right ACA territories, consistent with the results seen at angiography. The histograms of these regions (L1-L3 and R1-R3), while not as striking as those seen for Patient 2, nevertheless demonstrate significant increases in frequency of significantly mismatched pixels (e.g., shift of curve to the right for region L1).

The methods and systems described herein for quantifying symmetrical portions of an image may be used for purposes other than assisting in diagnosis of a patient based on an image. For example, the methods may be applied to compare a patient's image with prior images from that patient to observe progress over time or create a medical history for the patient. Also, the methods can be applied to train medical students in reading radiological images or to assess a physician's diagnostic abilities. The methods may be similarly applied in areas other than medical imaging, provided the image represents and captures a symmetrical body having characteristics expected to be symmetrically distributed about an axis.

The methods and systems described herein may be implemented in software, firmware, hardware, or any combination(s) of software, firmware, or hardware suitable for the purposes described herein. Software and other modules may reside on servers, workstations, personal computers, computerized tablets, PDAs, and other computer readable memory devices suitable for the purposes described herein. Software and other modules may be accessible via local memory, via a network, via a browser or other application in an ASP context, or via other means suitable for the purposes described herein. Data structures described herein may comprise computer files, variables, programming arrays, programming structures, or any electronic information storage schemes or methods, or any combinations thereof, suitable for the purposes described herein. User interface elements described herein may comprise elements from graphical user interfaces, command line interfaces, and other interfaces suitable for the purposes described herein. Screenshots presented and described herein can be displayed differently as known in the art to input, access, change, manipulate, modify, alter, and work with information.

While the invention has been described and illustrated in connection with preferred embodiments, many variations and modifications as will be evident to those skilled in this art may be made without departing from the spirit and scope of the invention, and the invention is thus not to be limited to the precise details of methodology or construction set forth above as such variations and modification are intended to be included within the scope of the invention.

1. A method for evaluating a medical image represented by image data, the method comprising:
assigning an axis of symmetry to the medical image;
computing, using the image data, at least one relative difference map based on a comparison of two substantially symmetrical areas around the axis of symmetry; and
generating a representation of any difference between the two symmetrical areas.

2. The method of claim 1, comprising scanning a region of interest to acquire the image data.

3. The method of claim 2, wherein scanning comprises performing a computed tomography scan.

4. The method of claim 1, wherein computing comprises generating at least one difference map.

5. The method of claim 1, wherein generating comprises generating a three dimensional color image illustrating the relative difference between the two substantially symmetrical areas.

6. The method of claim 1, wherein generating comprises generating a histogram representing the relative difference between the two substantially symmetrical areas.

7. The method of claim 1, wherein assigning comprises a user assigning the axis of symmetry through a user interface.

8. The method of claim 1, wherein assigning comprises automatically assigning the axis of symmetry based on the image data.

9. The method of claim 1, wherein computing comprises computing a statistical discrepancy between the two substantially symmetrical areas.

10. The method of claim 9, wherein computing comprises using the Kolmogorov-Smirnov test to compute the statistical discrepancy between the two substantially symmetrical areas.

11. The method of claim 9, comprising defining at least two windows in the image data, each window representing one of the symmetrical areas for which at least one relative difference map is to be computed.

12. The method of claim 11, wherein defining the windows comprises positioning each window in substantially equidistant locations from the assigned axis of symmetry.

13. The method of claim 11, wherein defining the windows comprises defining the windows as having n×n pixels of the image data.

14. The method of claim 13, where n=9.

15. The method of claim 1, comprising repeating the computing and generating steps for a second set of substantially symmetrical areas around the axis of symmetry to generate a second relative difference map.

16. A computer readable medium storing program code which, when executed, causes a computer to perform a method for evaluating a medical image represented by image data, the method comprising:
assigning an axis of symmetry to the medical image;
computing, using the image data, at least one relative difference map based on a comparison of two substantially symmetrical areas around the axis of symmetry; and
generating a representation of any difference between the two symmetrical areas.

17. A computer readable medium storing a data structure representing a relative difference map, the data structure comprising a quantification of statistical differences between image data values taken from corresponding value windows located substantially symmetrically with respect to an assigned axis of symmetry in a medical image.

18. A method for evaluating the symmetry of an image represented by image data, comprising:
computing a shape of a substantially symmetrical object of interest based on image data, the object of interest having at least two substantially symmetrical sections;
assigning an axis of symmetry to the object of interest such that the axis lies between the two substantially symmetrical sections;
optionally converting the shape of the object of interest to a substantially rectangular or square shape;
optionally normalizing the converted shape;
determining, using the image and shape information, a degree of symmetry between the at least two substantially symmetrical sections with respect to the axis of symmetry; and
generating a graphical representation of any difference between the two substantially symmetrical sections.

19. The method of claim 18 wherein the computing further comprises using a bounding function to compute the shape of the substantially symmetrical object of interest.

20. The method of claim 18 wherein the determining further comprises performing a pixel comparison of the image and shape information to determine the degree of symmetry.

21. The method of claim 18 wherein the computing further comprises using a Fourier shape descriptor to compute the shape of the substantially symmetrical object of interest.

22. The method of claim 18 wherein the assigning further comprises computing at least one centroid to define the axis of symmetry.
